(19) United States
(12) Patent Application Publication
(10) Pub. No.: US 2019/0093064 A1
(43) Pub. Date: Mar. 28, 2019

(54) Passive Acoustic Method of Determining Oxygen Production and Photosynthesis Rate of Algae

(71) Applicant: The Government of the United States of America, as represented by the Secretary of the Navy, Arlington, VA (US)

(72) Inventors: Simon E. Freeman, Portsmouth, RI (US); Lauren A. Freeman, Portsmouth, RI (US)

(21) Appl. No.: 16/147,081

(22) Filed: Sep. 28, 2018

Related U.S. Application Data
(60) Provisional application No. 62/564,563, filed on Sep. 28, 2017.

(51) Int. Cl. C12M 1/34; C12M 1/00

(52) U.S. Cl. CPC C12M 41/46; C12M 21/02; C12M 31/02

(57) ABSTRACT

A bioreactor having passive acoustic quantification of photosynthetic rate determination, comprising a tank for holding a fluid medium for photosynthetic bioreaction; one or more hydrophones located within the tank and configured to transduce passive acoustic signals within the tank into an electric signal and transmit that electric signal; a data acquisition and processor configured to receive the electric signal and calculate real-time productivity of oxygen by calculating the volume of oxygen produced using Minnaert frequency relationship.

### RELATED APPLICATIONS

This application claims the benefit of U.S. Provisional Application No. 62/564,563 filed Sep. 28, 2017, which is hereby incorporated herein by reference.

### FIELD OF INVENTION

The present invention relates generally to measuring the rate of photosynthesis performed by aquatic plants and algae, and more particularly to a passive acoustic method thereof.

### BACKGROUND

In applications such as an algal biofuel reactor, monitoring algal condition is conventionally performed by extracting a small algal sample and optically evaluating the chlorophyll content. Bulk quantification could be performed by weighing algae before and after exposure to Photosynthetically Available Radiation (PAR) and nutrients, but that approach does not enable continuous monitoring. Computer-vision-based optical image processing could provide continuous measurements, but the transparency of fluid within algal bioreactors can be too low for accurate volumetric estimates to be made using this technique.

In addition, the monitoring of algal condition is important in seaweed farming. In that application, monitoring is performed by visual inspection of the algae from a surface vessel, which is a time- and labor-intensive process. More recently, techniques are being developed that would enable underwater autonomous vehicle-based active acoustic survey of algae. These techniques are limited in that the cost of an underwater autonomous vehicle is high, there is the risk of vehicle loss associated with propeller entanglement in algae, the influence of active acoustic systems on local wildlife and the permitting process required to use such systems is an additional burden, and the limited endurance and swath of battery-powered active sonar systems. Furthermore, an active acoustic algae profiler measures physical parameters associated with algae (such as biomass/backscatter), rather than a variable that is the direct result of real-time primary productivity.

### SUMMARY OF INVENTION

Therefore, provided herein is a method enabling passive volumetrically integrative quantification of primary production from aquatic plants/algae on a continuous basis in a tank or reactor-style environment. The method involves the use of a hydrophone or hydrophone array deployed in a closed volume, filled with photosynthesizing aquatic algae. Passive acoustic data are recorded and processed to extract the sound produced by bubbles of gas emanating from the algae surface, a direct indicator of photosynthetic activity.

The foregoing and other features of the invention are hereinafter described in greater detail with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic of the layout of an exemplary bioreactor apparatus to sense primary productivity by aquatic plants/algae through passive acoustic means. More than one hydrophone and array processing algorithms may be used depending on the desired directionality of the acoustic measurements.

FIG. 2 shows two photographs of algae Gracilaria salicornia actively creating gas bubbles during photosynthesis. White arrows indicate the locations of larger bubbles. The scale bar in the top photo is 5 mm in length. A closer view of one gas bubble about to detach from the algae surface is shown in the lower photograph having a scale bar of 2 mm in length.

FIG. 3 shows a 9 s time series of ambient sound from inside an aquarium.

FIG. 4 shows a high-resolution view of a typical single transient bubble waveform as shown in FIG. 3.

FIG. 5 shows a spectrogram showing the time-varying spectral content of the received waveform in FIG. 4.

FIG. 6 shows a pressure spectral density (PSD) estimate showing mean, median and maximum pressure spectral densities of received level from the 2 ms period as shown in FIGS. 4 and 5, indicating the spectral peak of the waveform as 13.07 kHz (vertical dashed line).

FIG. 7 shows an averaged spectrum from transient sounds recorded over one hour overlaid with standing-wave resonant tank modes. Mode sums indicate the sum of mode numbers in the horizontal (length, width) and vertical (depth) directions.

FIG. 8 shows the acoustic response of algae to light over time in ten-minute averaged time-frequency histograms showing the distribution of Minnaert frequencies from bubbles produced by algae with the application and removal of PAR. The color scale indicates the number of bubble events acoustically characterized over a 10-min period, per frequency bin (195 Hz bin width).

FIG. 9 shows Sound Exposure Level (SEL, 10 min. averages, ±1 S.E., left axis) and dissolved oxygen concentration (right axis) showing an increase, decrease, and increase of dissolved oxygen and SEL with the application, removal and application of PAR, respectively. The grey regions indicate the period when the light source was removed.

FIG. 10A shows a log-linear regression plots of the number of bubbles per minute against dissolved oxygen levels (R²=0.76, exponential coefficients α=−2.54, β=0.43).

FIG. 10B shows the mean bubble radii per minute against dissolved oxygen levels (R²=0.47, exponential coefficients α=−3.85, β=0.19).

FIG. 10C shows 10-minute sound exposure level against dissolved oxygen levels (R²=0.82, exponential coefficients α=83.15, β=1.39).

FIG. 11A shows bubble size distributions obtained from photographically imaged measurements (histogram, left axis) and Minnaert radii (solid line, right axis) every 150 s over a one-hour period of active bubble formation.

FIG. 11B shows boxplots indicating 25th, 50th, 75th percentiles and 5th and 95th percentile outliers (crosses) of optically imaged and acoustically derived bubble radii distributions over the same period as FIG. 11A.

FIG. 12 shows an exemplary bioreactor.

FIG. 13 shows an exemplary bioreactor.

FIG. 14 shows an exemplary bioreactor.

### DETAILED DESCRIPTION

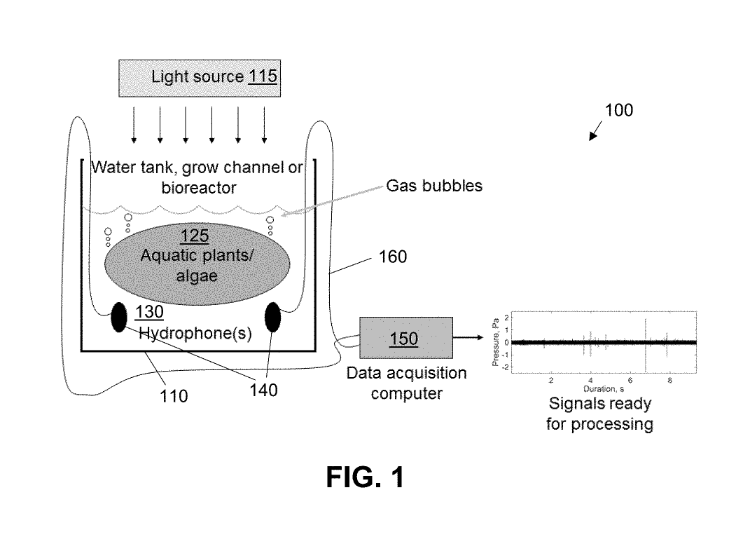

Passive acoustic quantification of algal photosynthetic rate involves the signal processing of bubble sound produced by waste gas emanating from the plant as underwater bubbles using an equipment setup similar to that shown in FIG. 1. FIG. 1 shows a schematic of the layout of an exemplary bioreactor apparatus 100 to sense primary productivity by aquatic plants/algae through passive acoustic means. More than one hydrophone and array processing algorithms may be used depending on the desired directionality of the acoustic measurements. The bioreactor 100 includes a main pool or tank 110, a light source 115, aquatic plants and/or algae 125 in the fluid medium (water) 130 and one or more hydrophones 140. The hydrophones may send their signals to a data acquisition and processor 150, by any appropriate means such as wirelessly or via cables 160, where real-time relative (or absolute, if calibrated) productivity is calculated using the method described below.

Algae release oxygen as a byproduct of photosynthesis. While these waste molecules are formed intracellularly in solution, the nucleation of oxygen gas bubbles on the surface of macroalgal tissue takes place when localized supersaturation of dissolved oxygen occurs at a nucleation site. Depending on the timescale of bubble formation and the total gas tension, the diffusion of nitrogen will also contribute to total bubble volume. Previous work has shown the causal relationship between oxygen bubble production by algae and oxygen supersaturation at the surface microenvironment. The bubbles grow with the addition of more waste oxygen produced by photosynthesis and the addition of nitrogen through diffusion, ultimately separating from the algae through a combination of buoyancy and surface tension forces. As a spherical bubble is perturbed through release, it oscillates with exponential decay at a frequency inversely proportional to its radius R₀, the roots of the specific heat ratio of the gas γ and ambient fluid pressure P_{fl}, and the inverse root of ambient fluid density, ρ_{fl}, a relationship first derived for generic bubble sources by Minnaert in 1933 and now referred to as the Minnaert frequency ω_{Minn}:

$${ω}_{Minn} = \frac{1}{{R}_{0}} \sqrt{\frac{3 γ {P}_{fi}}{{ρ}_{fl}}} \qquad ( 1 )$$

This approximation was first developed with the assumption of negligible heat flow (adiabatic conditions) and negligible surface tension, but has since been shown to be a good approximation for bubbles of radii between 30 nm to 300 μm. In alternative embodiments for some situations, it is possible that an adiabatic resonance frequency may produce a more accurate result by accounting for the effects of thermal diffusivity of gas inside the bubbles.

Consequently, passive acoustic estimates of bubble volume can be made if the water properties and depth at which bubble separation occurs are known.

The signal processing of acoustic time-series involves short-time Fourier analysis upon intensity filtered ambient noise recordings. This type of analysis provides a more spectrally detailed view of individual transient soundscape components because typical Fourier analysis approaches in underwater acoustics integrate over time periods that are much longer than each individual biological sound. While longer integration results in increased frequency resolution, ideal for detecting tonal and/or narrow-band sources, the approach can also spectrally smear multiple transient sounds together and/or reduce peak level estimates through giving equal weighting to quiet periods between transient arrivals. If persistent environmental noise is present, it can mask some transient biological sounds sampled through this approach. The approach associated with exemplary embodiments involves selecting each transient using an intensity filter, ensuring the transform length encompassed only the transient, and then assessing the spectral qualities of each transient individually.

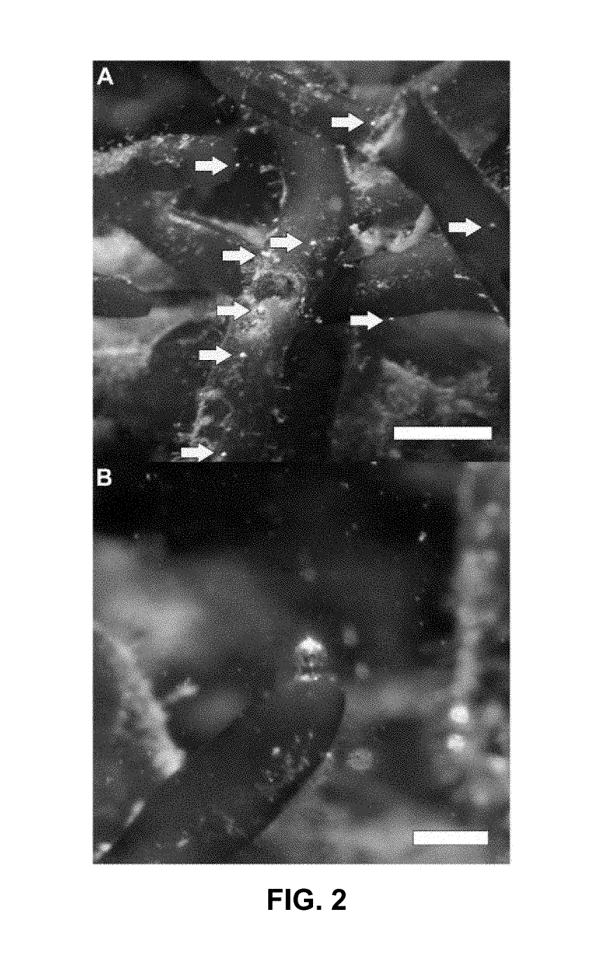

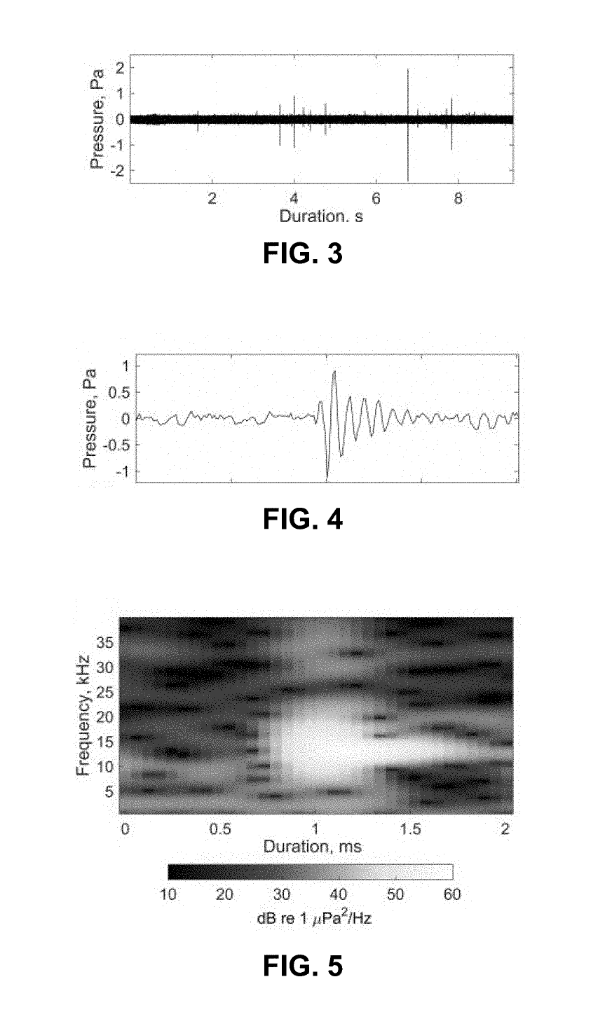

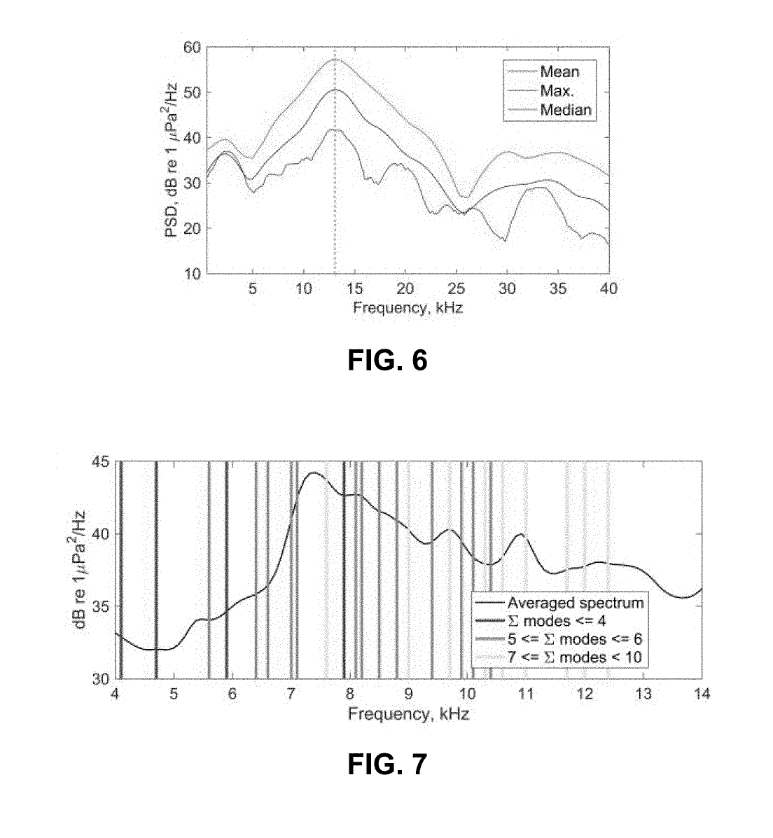

Bubbles can readily be observed with the naked eye on the surface of photosynthesizing macroalgae (FIG. 2). As the bubbles release from the plant, they create a short ‘ping’ sound. Acoustic recordings of bubble releases separated by quiet periods consequently appear as an irregular pulse-train-like time series (FIG. 3). The sound created by perturbation of the bubble during release is naturally transient and delays exponentially (FIG. 4). Oversampled recordings (100 kS·s⁻¹ in this case) permit more detailed spectral analysis of each sound (FIG. 5) and fundamental frequency estimates for each bubble can be made from the resultant data (FIG. 6). Tank resonance modes were restricted through the presence of algae and the drafted, acoustically absorptive nature of the tank walls and did not appear to substantially influence the recorded spectra (FIG. 7).

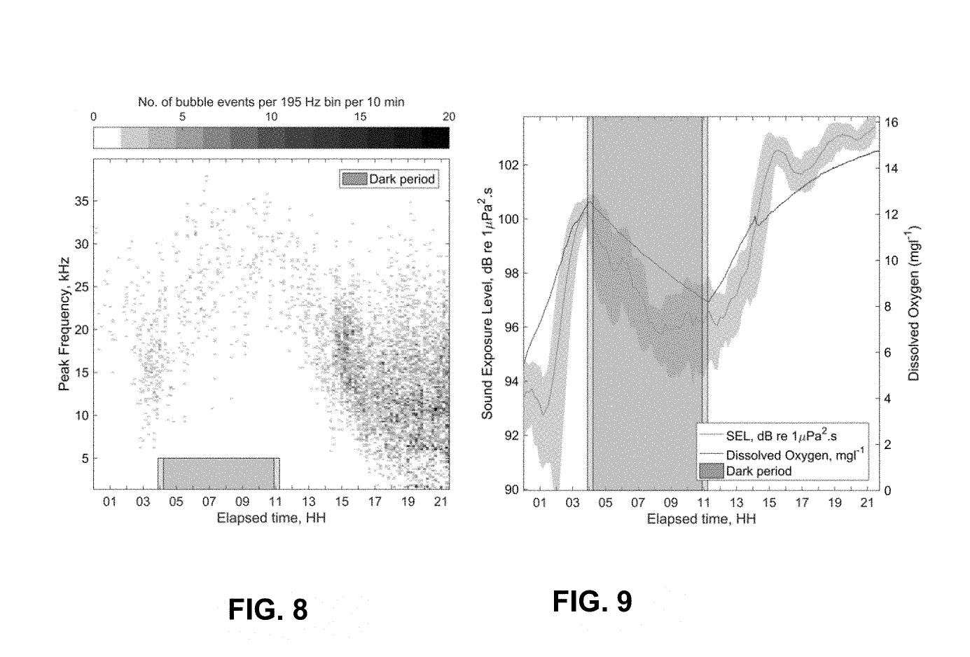

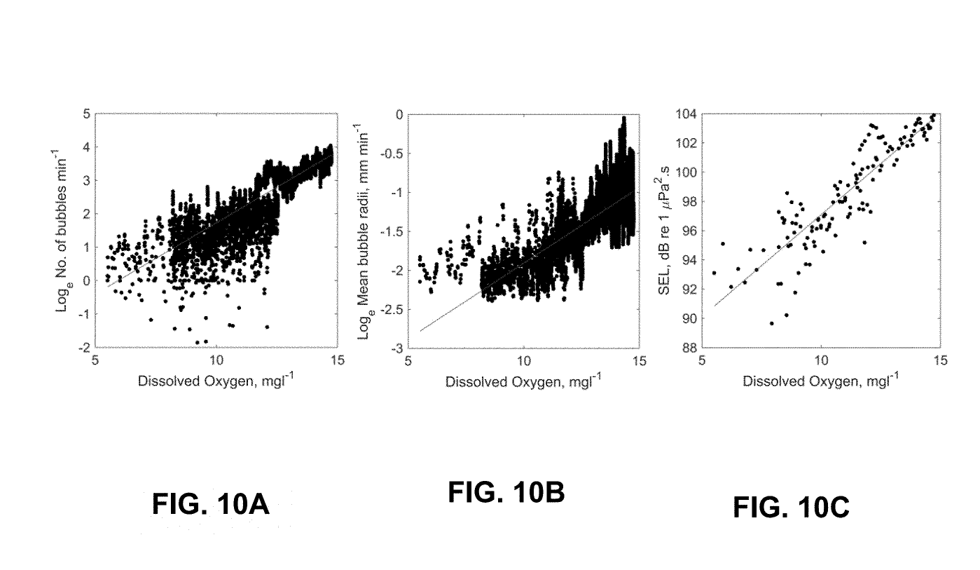

Experiments with filamentous algae, Salicornia gracilaria demonstrate that with the intermittent application of photosynthetically available radiation (PAR) the link between photosynthesis, dissolved oxygen and acoustic emissions through bubble formation becomes apparent (FIGS. 8 and 9). Over the duration of PAR application both the number of bubbles (R²=0.76, FIG. 10A) and their mean size (R²=0.47, FIG. 10B) increased with dissolved oxygen levels.

The process was reversed with the removal of light. These increases in the size and rate of bubble formation during the illuminated period lower the frequency distribution of sounds produced by the algae (FIG. 8) and cause an increase in the Sound Exposure Level (SEL, R²=0.82, FIG. 8 and FIG. 10C), a measure analogous to acoustic work.

Regarding FIGS. 10A-C, log-linear regression plots of acoustic emissions from photosynthesizing algae are shown. In FIG. 10A the number of bubbles per minute against dissolved oxygen levels (R²=0.76, exponential coefficients α=−2.54, β=0.43) is shown. In FIG. 10B mean bubble radii per minute against dissolved oxygen levels (R²=0.47, exponential coefficients α=−3.85, β=0.19) is shown. In FIG. 10C 10-minute Sound Exposure Level against dissolved oxygen levels (R²=0.82, exponential coefficients α=83.15, β=1.39). The coefficients may be applied to an exponential regression of linear parameters x and y as follows:

y=e^{a}·e^{βx} (2)

The onset of bubbling rate and size increase from the application of light was delayed due to the time required for photosynthesizing algae to build supersaturation conditions in the surrounding water, and for bubbles to reach a size large enough to create sufficient buoyancy force to detach from the algae. After the light source was removed, there was a similar delay in the reduction of bubble production and sound, due to bubbles remaining on the algae and saturation state remaining above 100 percent.

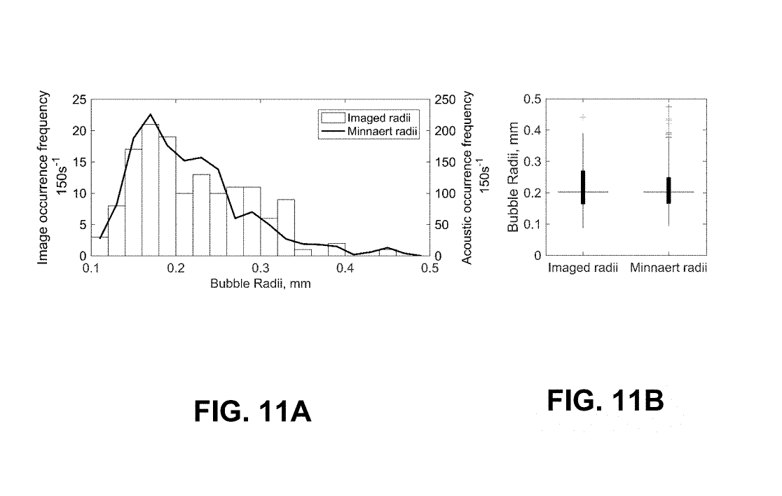

A comparison between acoustically derived bubble size distributions and photographically obtained measurements of bubble size (FIGS. 11A and B) optically validates the acoustic estimation of algae-driven bubble radii (99 percent significance no evidence the distributions were unequal).

The quantification of aquatic plant and algae through passive acoustic sensing is unprecedented. A properly calibrated system would enable a volumetrically integrative, remote and noninvasive method of measuring primary productivity outputs in semi-real-time, the slight delay being due to bubble formation preceding separation of the bubble from the plant/algae surface, signal processing computational burden and the requirement that ensemble averages are required in the depiction of some data.

Exemplary methods are superior to conventional optical methods of sampling algal state (i.e., optical quantification of chlorophyll content in a sample) as it can be performed in turbid water or through opaque tank walls, and is not affected by the opacity of the algae. This new technique samples the entire volume of fluid in which sound can propagate, meaning there are fewer constraints on tank/reactor vessel shape. Furthermore, it presents an advantage over optical sensing systems because the acoustic emissions are a direct result of primary productivity within the entire tank/reactor volume, rather than an indirect indicator such as the chlorophyll content in a small sample of algae or biomass estimates obtained through active acoustic return.

The simplest exemplary embodiment includes a single hydrophone placed inside a fluid volume containing aquatic plants/algae with at least one transparent or open side exposed to PAR. The hydrophone is connected to a data acquisition system that performs intensity-filtered spectral estimation of transient peak frequencies. An alternative method and apparatus for sampling sounds produced by photosynthesis is to use a hydrophone array. In a larger or more geometrically complex fluid container, an array of hydrophones may permit a higher signal-to-noise ratio (SNR) between bubble sound and background noise. Furthermore, the hydrophone array may permit spatial localization of individual bubble sounds or groups of sound, indicating any spatial variation of primary productivity within the tank volume (i.e., the array apparatus and method could indicate a region of the tank that may be inadvertently shaded from light, reducing output).

Because bubble production rates can be quantified through sufficiently sampled acoustic time-series, and that the size of each bubble is discernible from its ring by way of the Minnaert frequency, the quantification of oxygen production, and consequently glucose production, is also possible.

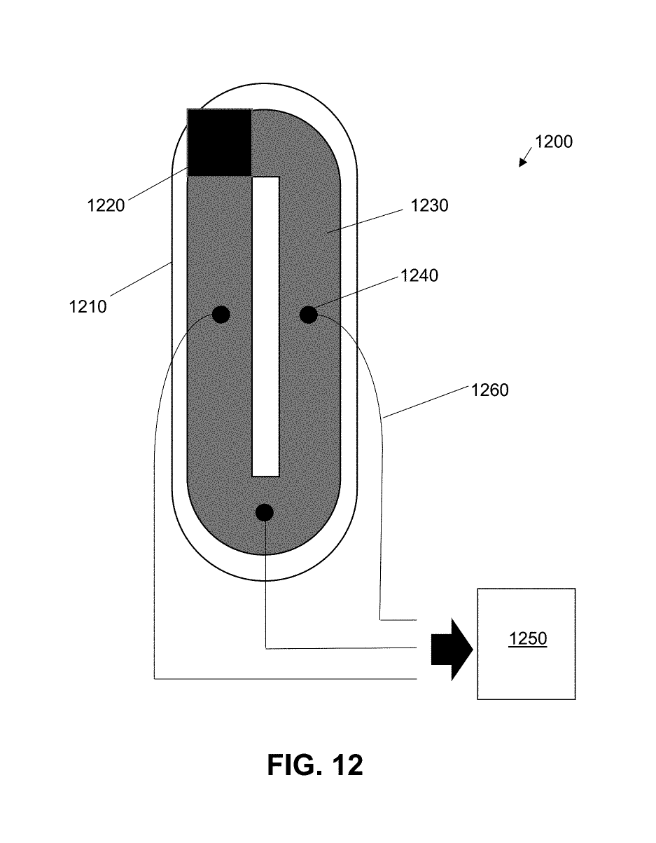

Turning now to FIG. 12, an exemplary embodiment of an outdoor horizontal algal bioreactor is shown at 1200. The bioreactor 1200 includes a main pool or tank 1210, a circulator 1220 for circulating the fluid medium (water) 1230, and one or more hydrophones 1240. The hydrophones may send their signals to a data acquisition and processor 1250, by any appropriate means such as wirelessly or via cables 1260, where real-time relative (or absolute, if calibrated) productivity is calculated using the method described above.

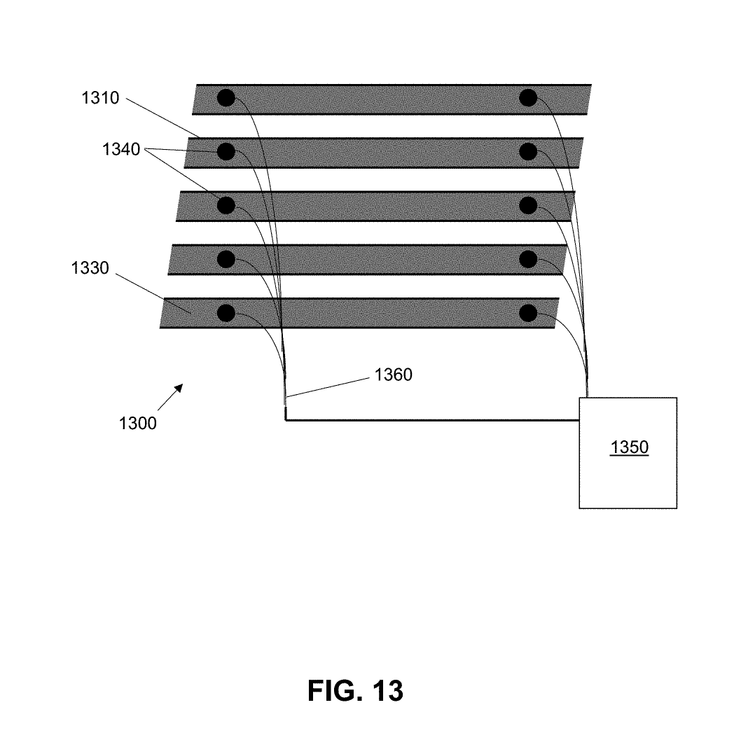

Turning now to FIG. 13, an exemplary embodiment of a tubular bioreactor is shown at 1300. The bioreactor 1300 is substantially the same as the above-referenced bioreactor 1200, and consequently the same reference numerals but indexed by 100 are used to denote structures corresponding to similar structures in the tubular bioreactor. In addition, the foregoing description of the bioreactor 1200 is equally applicable to the bioreactor 1300 except as noted below. Moreover, it will be appreciated upon reading and understanding the specification that aspects of the bioreactors may be substituted for one another or used in conjunction with one another where applicable.

Instead of or additional to an outdoor tank, one or more (typically dozens of serially and/or parallel connected) tube lengths 1310 may be used in a closed loop system that typically includes a pump, filter system, feeding vessel, etc. (not shown). In exemplary tubular bioreactors, an array of hydrophones 1340 may be used to acquire the acoustic signals needed to determine oxygen production. This array is preferably spaced at consistent distances along each tube length, although other configurations are possible.

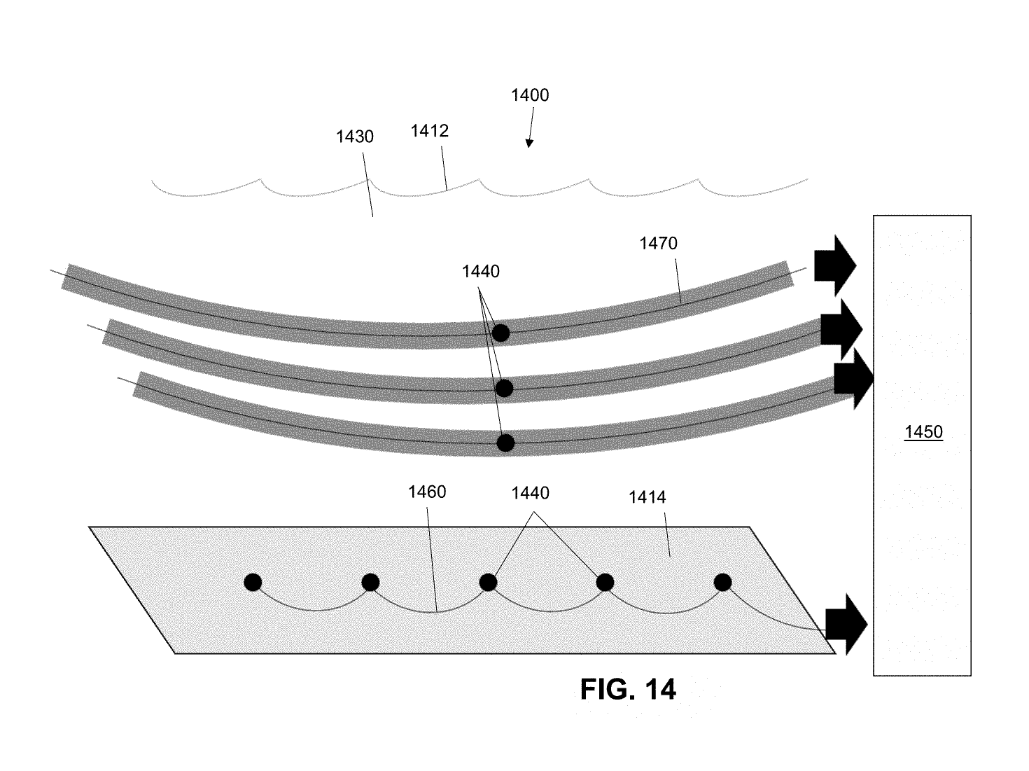

Turning now to FIG. 14, an exemplary embodiment of a more natural bio-reactor, a marine or fresh water algal farm, is shown at 1400. The algal farm 1400 is substantially the same as the above-referenced bioreactors, and consequently the same reference numerals but indexed by 100 are used to denote structures corresponding to similar structures in the algal farm. In addition, the foregoing description of the bioreactors are equally applicable to the algal farm except as noted below. Moreover, it will be appreciated upon reading and understanding the specification that aspects of the bioreactors may be substituted for one another or used in conjunction with one another where applicable.

Instead of a closed tank or tube set, an algal farm may be in an open, enclosed, or partially enclosed natural marine or freshwater (pond, lake) environment. Although the volume may be unbounded laterally, a top and bottom of the volume may be generally known by the surface of the water 1412 and the floor 1414 of the body of water. Algae may be preferably grown on one or more algae lines 1470 suspended in the farm 1400. One or more hydrophones 1440 may be placed independently of the algae lines 1470, or may be placed on the algae lines 1470.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A bioreactor having passive acoustic quantification of photosynthetic rate determination, comprising:
a tank for holding a fluid medium for photosynthetic bioreaction;
one or more hydrophones located within the tank and configured to transduce passive acoustic signals within the tank into an electric signal and transmit that electric signal;
a data acquisition and processor configured to receive the electric signal and calculate real-time productivity of oxygen by calculating the volume of oxygen produced using Minnaert frequency relationship.
